(19) United States
(12) Patent Application Publication
(10) Pub. No.: US 2024/0066357 A1
(43) Pub. Date: Feb. 29, 2024

(54) ATHLETIC PERFORMANCE TRAINING SYSTEM

(71) Applicant: SPELLMAN PERFORMANCE LLC, San Diego, CA (US)

(72) Inventor: LESTER SPELLMAN, San Diego, CA (US)

(21) Appl. No.: 18/453,860

(22) Filed: Aug. 22, 2023

Related U.S. Application Data
(60) Provisional application No. 63/400,568, filed on Aug. 24, 2022.

(51) Int. Cl. A63B 24/00; A63B 69/00

(52) U.S. Cl. CPC A63B 24/0075; A63B 24/0062; A63B 69/0028; A63B 2214/00; A63B 2220/12; A63B 2220/30; A63B 2220/40; A63B 2220/51; A63B 2220/806; A63B 2220/836

(57) ABSTRACT

Systems, apparatuses, and methods for improving an athlete's performance are described. A method includes receiving a force velocity profile (FVP) of a user (e.g., an athlete). Based the FVP, the user is assigned to a group of a plurality of groups. A load profile of the user is generated and velocity changes for different load conditions are determined. The load profile is indicative of a decrease in velocity of the user for increased load conditions. Based on the grouping and the load profile of the user, a training program specific to the user is determined. Further, the user may be instructed to perform a physical activity of the one or more physical activities from the training program.

### CROSS-REFERENCE TO RELATED APPLICATION

This application claims priority to U.S. Provisional Patent Application No. 63/400,568, entitled ATHLETIC PERFORMANCE TRAINING SYSTEM, filed Aug. 24, 2022, the contents of which are hereby incorporated in their entirety by this reference.

### TECHNICAL FIELD

This description relates to improving systems and methods related to training of athletes.

### BACKGROUND

Improving athlete performance often involves data collection using sensors. An athlete's performance is determined by analyzing the sensor data. Several performance improvement programs focus mainly on the quality of the athlete's movement and/or the athlete's ability to acquire a skill. Collecting more data enables tracking of more variables to better understand what a training program accomplishes. However, there is a need to not only collect relevant data, but also interpret data to generate actionable training instructions for customizing training programs and improving athlete performance.

### SUMMARY

In an embodiment, there are provided systems and methods for determining an athlete's performance and programs for improving their performance. For example, a method includes receiving a force velocity profile (FVP) of a user, and a plurality of groups characterized by a force ratio of forces exerted during the running activity, the force velocity profile being a relationship between force and velocity associated the user during a running activity; assigning, based on the FVP, the user in a group of the plurality of groups; generating a load profile of the user by changing load conditions of the user during the running activity and analyzing velocity changes for the load condition, the load profile indicative of a decrease in velocity of the user for different load conditions; determining, based on the grouping and the load profile of the user, a training program to improve performance of the running activity of the user, the training program comprising one or more physical activities to be performed for a specified number of times using specified weights; and instructing the user to perform a physical activity of the one or more physical activities from the training program.

According to an embodiment, there is provided a computer system comprising a non-transitory computer readable medium having instructions recorded thereon. The instructions, when executed by a computer, implement the method steps above.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed embodiments. In the drawings,

FIG. 1 illustrates a block diagram of a system for training a user (e.g., an athlete), according to an embodiment.

FIG. 2 illustrates a sample grouping of the user based on a force velocity profile, according to an embodiment.

FIG. 3 illustrates a sample load profile and training programs, according to an embodiment.

FIG. 4 is a graphical representation of the load profile of FIG. 3, according to an embodiment.

FIG. 5 illustrates instructions corresponding to the load profile of the user, according to an embodiment.

FIG. 6 illustrates a training system, according to an embodiment.

FIG. 7 illustrates a flow chart of an exemplary method for determining a training program for the user (e.g., an athlete), according to an embodiment.

FIG. 8 is a block diagram of an example computer system, according to an embodiment.

### DETAILED DESCRIPTION

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed embodiment(s). However, it will be apparent to those skilled in the art that the disclosed embodiment(s) may be practiced without those specific details. In some instances, well-known structures and components may be shown in block diagram form in order to avoid obscuring the concepts of the disclosed subject matter.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

Various other aspects, features, and advantages of the inventions will be apparent through the detailed description of the invention and the drawings attached hereto. It is also to be understood that both the foregoing general description and the following detailed description are exemplary and not restrictive of the scope of the inventions. As used in the specification and in the claims, the singular forms of “a”, “an”, and “the” include plural referents unless the context clearly dictates otherwise. In addition, as used in the specification and the claims, the term “or” means “and/or” unless the context clearly dictates otherwise.

As used herein, unless specifically stated otherwise, the term “or” encompasses all possible combinations, except where infeasible. For example, if it is stated that a database can include A or B, then, unless specifically stated otherwise or infeasible, the database can include A, or B, or A and B. As a second example, if it is stated that a database can include A, B, or C, then, unless specifically stated otherwise or infeasible, the database can include A, or B, or C, or A and B, or A and C, or B and C, or A and B and C.

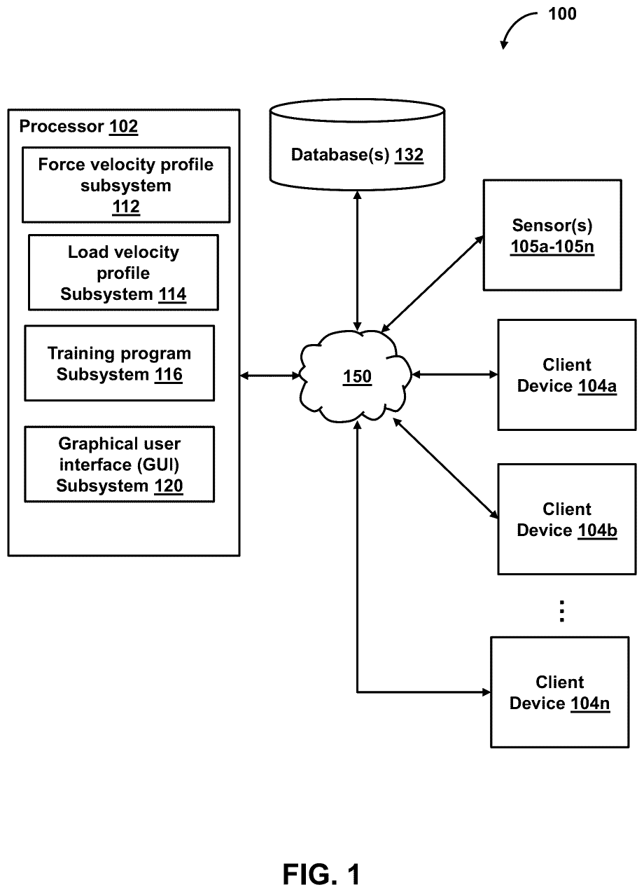

FIG. 1 shows a system 100 for determining user (e.g., athlete) performance and training the user (e.g., the athlete), in accordance with one or more embodiments. As shown in FIG. 1, system 100 may include computer system 102, client device 104 (client devices 104a-104n), one or more sensors 105a-105n, or other components. Computer system 102 may include configuration force velocity profile subsystem 112, load velocity profile subsystem 114, training program subsystem 116, graphical user interface (GUI) subsystem 120, or other components. By the way of example, computer system 102 may include a distributed system, a cloud-based system, a container environment on one or more servers, or other systems. Each client device 104 may include any type of mobile terminal, fixed terminal, or other device. By the way of example, client device 104 may include any computing device, such as a personal computer (PC), a laptop computer, a tablet computer, a hand-held computer, a smartphone, or other computer equipment. Users may, for instance, utilize one or more client devices 104 to interact with one another, one or more servers, or other components of system 100. In an embodiment, a user (e.g., software developer, a trainer, an athlete, or other user) may interact with the GUI, using client device 104a, to view or display training programs, athlete's performance, force velocity profile, load velocity profile, or other outputs of the operations discussed herein. Such information may be stored/retrieved by computer system 102 in/from a storage system (e.g., database 132).

A component of system 100 may communicate with one or more components of system 100 via a communication network 150 (e.g., the Internet, a mobile phone network, a mobile voice or data network, a cable network, a public switched telephone network, or other types of communications networks or combinations of communications networks). The communication network may be a wireless or wired network.

It should be noted that, while one or more operations are described herein as being performed by particular components of system 100, those operations may, in some embodiments, be performed by other components of system 100. As an example, while one or more operations are described herein as being performed by components of computer system 102, those operations may, in some embodiments, be performed by components of client device 104.

In an embodiment, the force velocity profile subsystem 112 may be configured to generate or receive a force velocity profile (FVP) of a user. In an embodiment, one or more sensors capture signals related to a running activity of the user. Based on the captured signals the FVP profile may be generated. In an embodiment, the one or more sensors 105a-105n may be a global positioning sensor on the user, a video camera, a wearable device configured to gather speed, acceleration or force data of the user during the running activity, or other sensors. As such, the FVP may be obtained based on data acquired via a global positional sensor (GPS), video, speed sensors, or other wearable devices configured to capture forces and velocity related information.

In an embodiment, the force velocity profile may be a relationship between force and velocity associated the user during a running activity. For example, the FVP may be represented as a mathematical function of force and velocity, a table characterizing the relationship between the force and velocity, a graph, or other representations.

In an embodiment, the force velocity profile subsystem 112 receives the FVP of the user, and a plurality of groups characterized by a force ratio of forces exerted during the running activity. In an embodiment, the force ratio may be a ratio of a horizontal force and a vertical force exerted by the user during the running activity. Based on the FVP, the user may be assigned to a group of the plurality of groups.

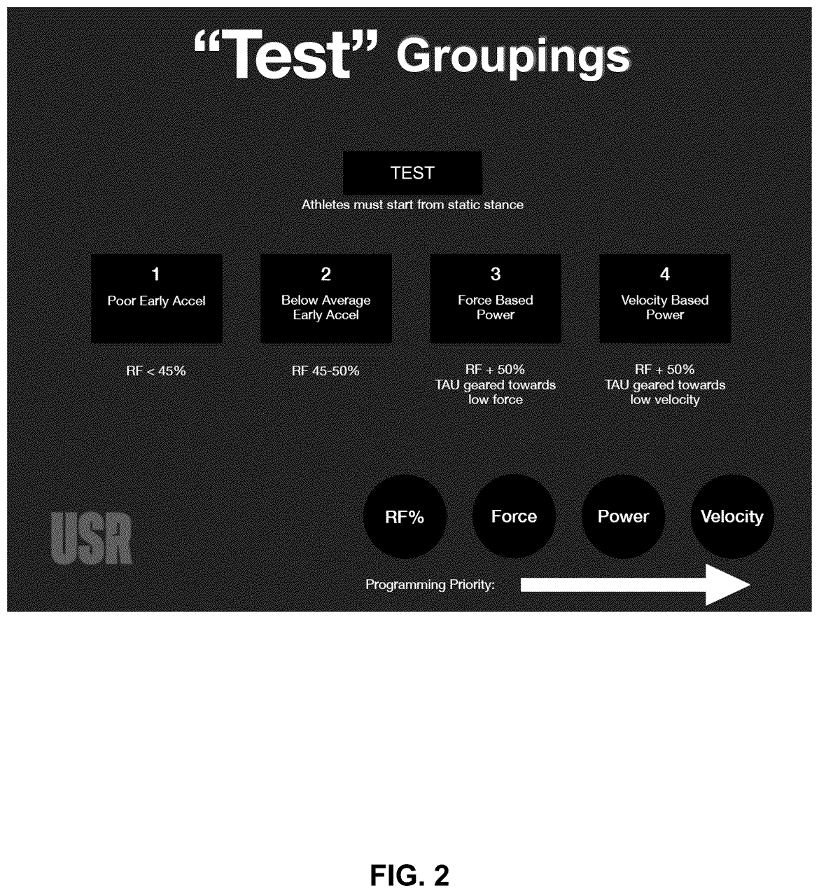

FIG. 2 illustrates exemplary groups associated with an FVP, according to an embodiment. As an example, an athlete's acceleration data over a period of time may be measured via the one or more sensors 105a-105n. For example, the acceleration data is measured from a start of the running activity to a peak velocity of the running activity. The acceleration data indicates how an athlete reaches a top speed.

In an embodiment, the acceleration data is further interpreted based on the FVP to determine which group a particular athlete should be assigned to. As shown in FIG. 2, the athletes may be assigned into one or the four different groups or categories. The values associated with the FVP used for grouping or categorizing the athlete are only exemplary. A person of ordinary skill in the art may select other values or ranges. The present disclosure is not limited to particular values or ranges for grouping based on FVP.

A first group may correspond to a force ratio value of less or equal to 45%. The first group indicates a substantially below average acceleration at the start of the running activity. In the present example, the first group has poor force ratio. For example, a force ratio of horizontal force to vertical force is a measure of how well the athlete's early acceleration is, and if it's poor (e.g., less than 45%), then the athlete is assigned to the first group.

A second group may correspond to a force ratio value between 45% to 50%. The second group is indicative of a below average acceleration at the start of the running activity.

A third group may correspond to a force ratio value of more than 50% and approximately 50% of a tau ratio, indicating a low force. In an embodiment, the tau ratio may be calculated as a ratio of acceleration to velocity. For example, the tau ratio is a peak acceleration divided by velocity. When the tau ratio is too low (e.g., below a certain threshold), it indicates that the athlete may not be producing enough acceleration. If the tau ratio is high (e.g., above a certain threshold), it indicates that the athlete may not be producing enough velocity.

The third group indicates that a force-based power is generated at the start of the running activity. The third group indicates an athlete has a good early acceleration, but the athlete may be breaking down and not able to maintain high levels of horizontal force as velocity increases. Thus, the athlete accelerates well early, but they're not able to maintain that acceleration rate. In an embodiment, the high levels of acceleration may be determined until they reach maximum velocity.

A fourth group is associated with a force ratio value of more than 50% and an approximately 50% tau ratio, indicating a low velocity. In an embodiment, the fourth group indicates a velocity-based power generated at the start of the running activity. The fourth group indicates the athlete can maintain a lot of acceleration as they get to a velocity, but this group does not reach a high enough velocity. As such, athletes in the third group may need to work on reaching a higher velocity.

Referring back to FIG. 1, the load velocity profile subsystem 114 may be configured to generate a load profile of the user by changing load conditions of the user during the running activity (e.g., a second round of the running activity) and analyzing velocity changes for the load condition. The load profile may be indicative of a decrease in velocity of the user for different (e.g., increasing) load conditions.

In an embodiment, determining the load profile involves instructing the user to slide (e.g., push or drag) a first load, a second load, a third load, or a fourth load, during the running activity, each load being characterized by a percentage of the body weight of the user.

The training program subsystem 116 may be configured to determine, based on the grouping and the load profile of the user, a training program to improve performance of the running activity of the user, the training program comprising one or more physical activities to be performed for a specified number of times using specified weights.

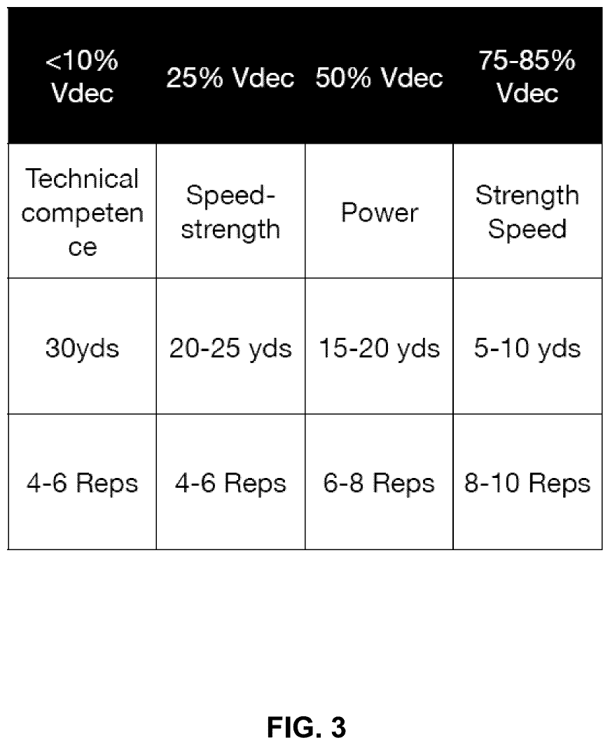

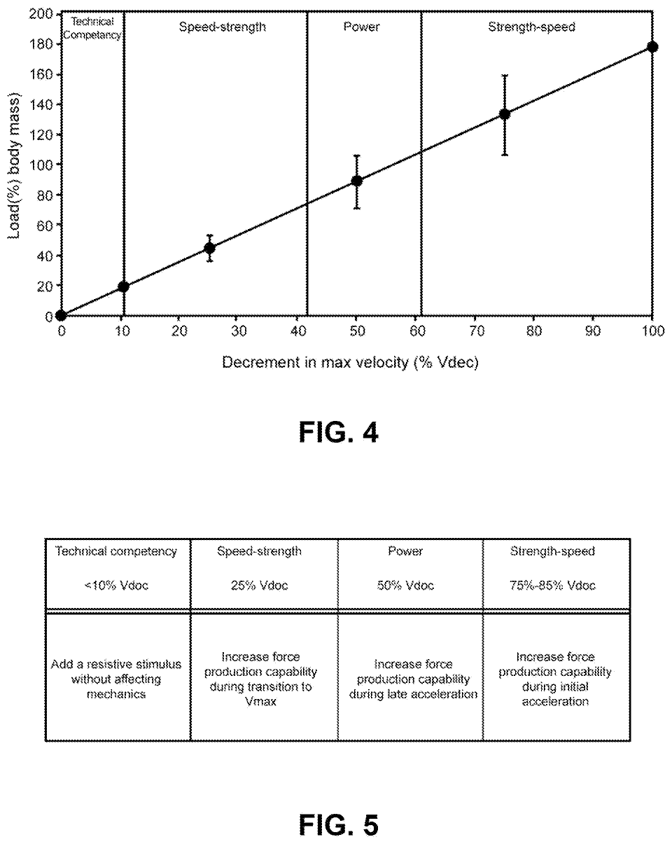

FIGS. 3-5 illustrates example load velocity profile and training programs. As shown in FIG. 4, for a given load velocity profile, athletes may run with four different loads (e.g., resisted guide, slide load, etc. attached to, pushed, or pulled by the athlete). For example, as described above, the loads may be a certain percentage of the athlete's body weight. The load values used herein are only exemplary. The present disclosure is not limited to any particular load values. A person of ordinary skill in the art may use different load values.

By way of a non-limiting example, the load profile may be determined by instructing the user to slide a load of approximately 10% of the body weight during the running activity; instructing the user to slide a load in a range between 10% to 25% of the body weight during the running activity; instructing the user to slide a load in a range between 25% to 50% of the body weight during the running activity; and instructing the user to slide a load in a range between 75% to 85% of the body weight during the running activity.

While the athlete is running with these loads, the velocity for each load is tracked. For example, when an athlete runs with 25% body weight, the velocity that the athlete reaches is observed. Then, a trend line is drawn on the velocity data to identify a percent of velocity decrease compared to when the athlete runs with no load conditions (i.e., only body weight with no additional weight or resistance added). In an example, for a 10% decrease in the velocity, a load that matches up with a 10% reduction in velocity (Vdec) is determined. For example, if the athlete runs at 20 mph, a load corresponding to a 10% reduction in velocity is determined. Similarly, determinations of load or velocity reductions may be made for 25%, 50% and 75% of body weight loads.

The training program subsystem 116 determines that if the athlete is in a particular group (e.g., the first group), then the athlete is recommended to perform one or more activities for strength. For example, an athlete in the first group may be assigned a training program in the speed strength category. If the athlete is in the second group, a training program corresponding to 50% Vdec (in FIG. 3) may be assigned.

In FIG. 3, the training program indicates the distances (in yards) and a number of repetitions (reps) to be performed by the athlete. For example, for an athlete in the first group in the strength category, it would be recommended to perform 8 to 10 reps of 5 to 10 yards with a load of 75% of the body weight. Accordingly, the training program is expected to improve whichever physical skill the athlete lacks. For example, being grouped in the first group indicates that an athlete lacks strength, so strength training may be recommended to improve the initial acceleration of the athlete.

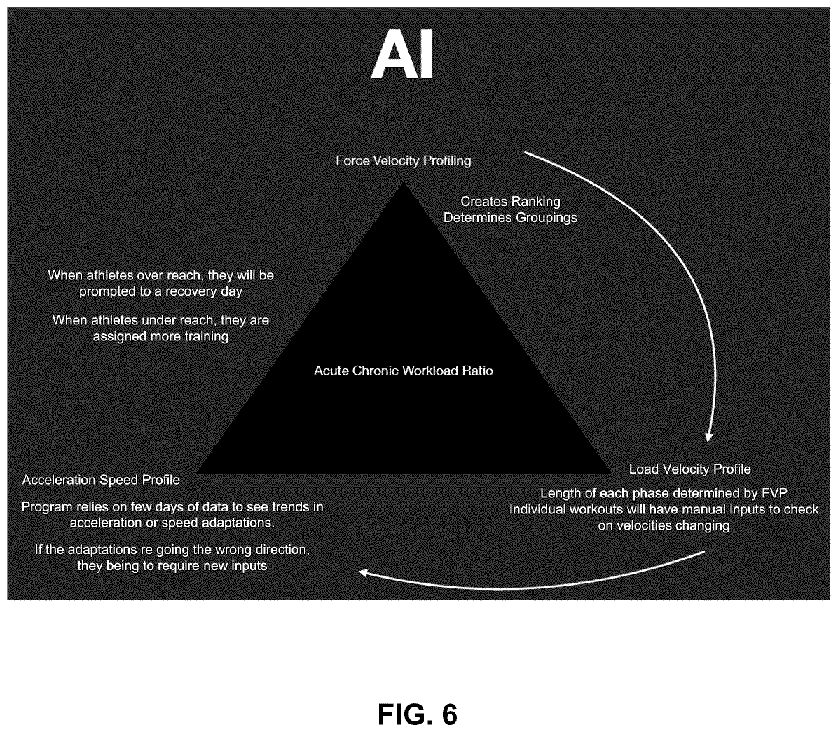

FIG. 6 illustrates the training system, according to an embodiment. The values used in the training system are only exemplary and do not limit the scope of the present disclosure. For example, a GPS may be attached to an athlete or a user and asked to run. The athlete may be asked to perform a run by pulling a load on a sled for e.g., 30 or 40 yard run. For this run, FVP data may be collected from the GPS. Additional data may be collected by asking the athlete to run with 25%, 50%, and 75% body weight on a sled. Using the FVP data from the four runs, a grouping and training program may be determined.

After completing the training program, for example, after four to five weeks of training according to a recommended training program, a regrouping may be performed and a different training program may be determined.

In an embodiment, acceleration speed profile data may be determined by accessing a cloud of data and drawing a trend line on the peak values. For example, data may be collected for 150 sprints or more. Depending on whether the trendline changes to positive or negative, the acceleration speed profile may indicate more training may be recommended on the acceleration side or on the speed side.

### Example Flowchart(s)

The example flowchart(s) described herein of processing operations of methods that enable the various features and functionality of the system as described in detail above. The processing operations of each method presented below are intended to be illustrative and non-limiting. In some embodiments, for example, the methods may be accomplished with one or more additional operations not described, and/or without one or more of the operations discussed. Additionally, the order in which the processing operations of the methods are illustrated (and described below) is not intended to be limiting.

In some embodiments, the methods may be implemented in one or more processing devices (e.g., a digital processor, an analog processor, a digital circuit designed to process information, an analog circuit designed to process information, a state machine, and/or other mechanisms for electronically processing information). The processing devices may include one or more devices executing some or all of the operations of the methods in response to instructions stored electronically on an electronic storage medium. The processing devices may include one or more devices configured through hardware, firmware, and/or software to be specifically designed for execution of one or more of the operations of the methods.

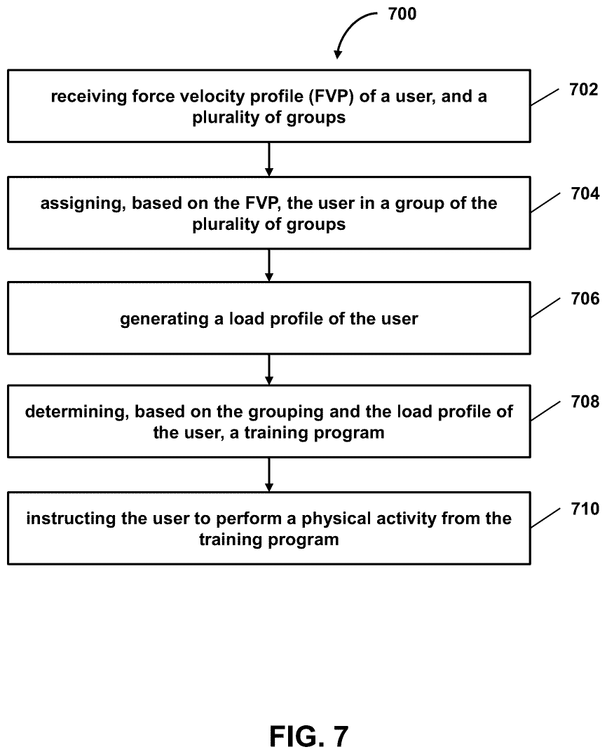

FIG. 7 illustrates an exemplary flow chart of the method for determining a training program for the user (e.g., an athlete), according to an embodiment. The method 700 may be implemented as processes or operations 702-710.

Process 702 may involve receiving force velocity profile (FVP) of a user, and a plurality of groups characterized by a force ratio of forces exerted during the running activity, the force velocity profile being a relationship between force and velocity associated the user during a running activity. The process 702 may be performed by the force velocity profile subsystem 112.

Process 704 may involve assigning, based on the FVP, the user in a group of the plurality of groups. The process 704 may be performed by the force velocity profile subsystem 112.

Process 706 may involve generating a load profile of the user by changing load conditions of the user during the running activity and analyzing velocity changes for the load condition, the load profile indicative of a decrease in velocity of the user for different load conditions. The process 706 may be performed by the load velocity profile subsystem 114.

Process 708 may involve determining, based on the grouping and the load profile of the user, a training program to improve performance of the running activity of the user, the training program comprising one or more physical activities to be performed for a specified number of times using specified weights. The process 708 may be performed by the training program subsystem 116.

Process 710 may involve instructing the user to perform a physical activity of the one or more physical activities from the training program. The process 710 may be performed by the GUI subsystem 120.

In some embodiments, the various computers and subsystems illustrated in FIG. 1 may include one or more computing devices that are programmed to perform the functions described herein. The computing devices may include one or more electronic storages (e.g., database(s) 132 or other electronic storages), one or more physical processors programmed with one or more computer program instructions, and/or other components. The computing devices may include communication lines or ports to enable the exchange of information within a network (e.g., network 150) or other computing platforms via wired or wireless techniques (e.g., Ethernet, fiber optics, coaxial cable, Wi-Fi, Bluetooth, near field communication, or other technologies). The computing devices may include a plurality of hardware, software, and/or firmware components operating together. For example, the computing devices may be implemented by a cloud of computing platforms operating together as the computing devices.

The electronic storages may include non-transitory storage media that electronically stores information. The storage media of the electronic storages may include one or both of (i) system storage that is provided integrally (e.g., substantially non-removable) with servers or client devices or (ii) removable storage that is removably connectable to the servers or client devices via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storage may store software algorithms, information determined by the processors, information obtained from servers, information obtained from client devices, or other information that enables the functionality as described herein.

The processors may be programmed to provide information processing capabilities in the computing devices. As such, the processors may include one or more of a digital processor, an analog processor, a digital circuit designed to process information, an analog circuit designed to process information, a state machine, and/or other mechanisms for electronically processing information. In some embodiments, the processors may include a plurality of processing units. These processing units may be physically located within the same device, or the processors may represent processing functionality of a plurality of devices operating in coordination. The processors may be programmed to execute computer program instructions to perform functions described herein of subsystems 112-120 or other subsystems. The processors may be programmed to execute computer program instructions by software; hardware; firmware; some combination of software, hardware, or firmware; and/or other mechanisms for configuring processing capabilities on the processors.

It should be appreciated that the description of the functionality provided by the different subsystems 112-120 described herein is for illustrative purposes, and is not intended to be limiting, as any of subsystems 112-120 may provide more or less functionality than is described. For example, one or more of subsystems 112-120 may be eliminated, and some or all of its functionality may be provided by other ones of subsystems 112-120. As another example, additional subsystems may be programmed to perform some or all of the functionality attributed herein to one of subsystems 112-120.

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

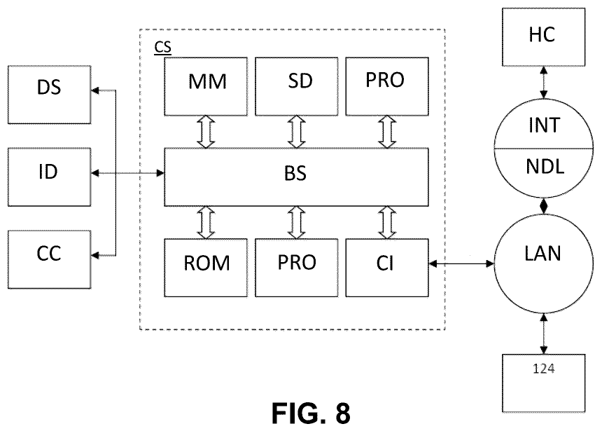

FIG. 8 is a block diagram of an example computer system CS, according to an embodiment. Computer system CS includes a bus BS or other communication mechanism for communicating information, and a processor PRO (or multiple processor) coupled with bus BS for processing information. Computer system CS also includes a main memory MM, such as a random access memory (RAM) or other dynamic storage device, coupled to bus BS for storing information and instructions to be executed by processor PRO. Main memory MM also may be used for storing temporary variables or other intermediate information during execution of instructions to be executed by processor PRO. Computer system CS further includes a read only memory (ROM) ROM or other static storage device coupled to bus BS for storing static information and instructions for processor PRO. A storage device SD, such as a magnetic disk or optical disk, is provided and coupled to bus BS for storing information and instructions.

Computer system CS may be coupled via bus BS to a display DS, such as a cathode ray tube (CRT) or flat panel or touch panel display for displaying information to a computer user. An input device ID, including alphanumeric and other keys, is coupled to bus BS for communicating information and command selections to processor PRO. Another type of user input device is cursor control CC, such as a mouse, a trackball, or cursor direction keys for communicating direction information and command selections to processor PRO and for controlling cursor movement on display DS. This input device typically has two degrees of freedom in two axes, a first axis (e.g., x) and a second axis (e.g., y), that allows the device to specify positions in a plane. A touch panel (screen) display may also be used as an input device.

According to one embodiment, portions of one or more methods described herein may be performed by computer system CS in response to processor PRO executing one or more sequences of one or more instructions contained in main memory MM. Such instructions may be read into main memory MM from another computer-readable medium, such as storage device SD. Execution of the sequences of instructions contained in main memory MM causes processor PRO to perform the process steps described herein. One or more processors in a multi-processing arrangement may also be employed to execute the sequences of instructions contained in main memory MM. In an alternative embodiment, hard-wired circuitry may be used in place of or in combination with software instructions. Thus, the description herein is not limited to any specific combination of hardware circuitry and software.

The term “computer-readable medium” as used herein refers to any medium that participates in providing instructions to processor PRO for execution. Such a medium may take many forms, including but not limited to, non-volatile media, volatile media, and transmission media. Non-volatile media include, for example, optical or magnetic disks, such as storage device SD. Volatile media include dynamic memory, such as main memory MM. Transmission media include coaxial cables, copper wire and fiber optics, including the wires that comprise bus BS. Transmission media can also take the form of acoustic or light waves, such as those generated during radio frequency (RF) and infrared (IR) data communications. Computer-readable media can be non-transitory, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, and EPROM, a FLASH-EPROM, any other memory chip or cartridge. Non-transitory computer readable media can have instructions recorded thereon. The instructions, when executed by a computer, can implement any of the features described herein. Transitory computer-readable media can include a carrier wave or other propagating electromagnetic signal.

Various forms of computer readable media may be involved in carrying one or more sequences of one or more instructions to processor PRO for execution. For example, the instructions may initially be borne on a magnetic disk of a remote computer. The remote computer can load the instructions into its dynamic memory and send the instructions over a telephone line using a modem. A modem local to computer system CS can receive the data on the telephone line and use an infrared transmitter to convert the data to an infrared signal. An infrared detector coupled to bus BS can receive the data carried in the infrared signal and place the data on bus BS. Bus BS carries the data to main memory MM, from which processor PRO retrieves and executes the instructions. The instructions received by main memory MM may optionally be stored on storage device SD either before or after execution by processor PRO.

Computer system CS may also include a communication interface CI coupled to bus BS. Communication interface CI provides a two-way data communication coupling to a network link NDL that is connected to a local network LAN. For example, communication interface CI may be an integrated services digital network (ISDN) card or a modem to provide a data communication connection to a corresponding type of telephone line. As another example, communication interface CI may be a local area network (LAN) card to provide a data communication connection to a compatible LAN. Wireless links may also be implemented. In any such implementation, communication interface CI sends and receives electrical, electromagnetic or optical signals that carry digital data streams representing various types of information.

Network link NDL typically provides data communication through one or more networks to other data devices. For example, network link NDL may provide a connection through local network LAN to a host computer HC. This can include data communication services provided through the worldwide packet data communication network, now commonly referred to as the “Internet” INT. Local network LAN (Internet) both use electrical, electromagnetic or optical signals that carry digital data streams. The signals through the various networks and the signals on network data link NDL and through communication interface CI, which carry the digital data to and from computer system CS, are exemplary forms of carrier waves transporting the information.

Computer system CS can send messages and receive data, including program code, through the network(s), network data link NDL, and communication interface CI. In the Internet example, host computer HC might transmit a requested code for an application program through Internet INT, network data link NDL, local network LAN and communication interface CI. One such downloaded application may provide all or part of a method described herein, for example. The received code may be executed by processor PRO as it is received, and/or stored in storage device SD, or other non-volatile storage for later execution. In this manner, computer system CS may obtain application code in the form of a carrier wave.

The descriptions above are intended to be illustrative, not limiting. Thus, it will be apparent to one skilled in the art that modifications may be made as described without departing from the scope of the claims set out below.

What is claimed is:

1. A system for training a user, the system comprising:
one or more sensors configured to output a force velocity profile (FVP) of a user, the force velocity profile being a relationship between force and velocity associated the user during a running activity; and
one or more processors configured to:
receive the FVP of the user, and a plurality of groups characterized by a force ratio of forces exerted during the running activity;
assign, based on the FVP, the user to a group of the plurality of groups;
generate a load profile of the user by changing load conditions of the user during the running activity and analyzing velocity changes for the load condition, the load profile indicative of a decrease in velocity of the user for different load conditions;
determine, based on the grouping and the load profile of the user, a training program to improve performance of the running activity of the user, the training program comprising one or more physical activities to be performed for a specified number of times using specified weights; and
instruct the user to perform a physical activity of the one or more physical activities from the training program.

2. The system of claim 1, wherein the force ratio is a ratio of a horizontal force and a vertical force exerted by the user during the running activity.

3. The system of claim 1, wherein the plurality of groups comprises:
a first group having the force ratio value of less or equal to 45%, the first group indicative of a substantially below average acceleration at the start of the running activity;
a second group having the force ratio value between 45% to 50%, the second group indicative of a below average acceleration at the start of the running activity;
a third group having the force ratio value of more than 50% and approximately 50% of an acceleration-velocity ratio indicating a low force, the third group indicative of a force-based power generated at the start of the running activity; and
a fourth group having the force ratio value of more than 50% and approximately 50% of an acceleration-velocity ratio indicating a low velocity, the fourth group indicative of a velocity-based power generated at the start of the running activity.

4. The system of claim 1, wherein determining the load profile comprises:
instructing the user to slide a first load, a second load, a third load, or a fourth load, during the running activity, each load being characterized by a percentage of the body weight of the user.

5. The system of claim 4, wherein determining the load profile comprises:
instructing the user to slide a load of approximately 10% of the body weight during the running activity;
instructing the user to slide a load in a range between 10% to 25% of the body weight during the running activity;
instructing the user to slide a load in a range between 25% to 50% of the body weight during the running activity; and
instructing the user to slide a load in a range between 75% to 85% of the body weight during the running activity.

6. The system of claim 1, wherein the one or more sensors comprises:
a global positioning sensor on the user;
a video camera; or
a wearable device configured to gather speed, acceleration or force data of the user during the running activity.

7. A method comprising:
receiving force velocity profile (FVP) of a user, and a plurality of groups characterized by a force ratio of forces exerted during the running activity, the force velocity profile being a relationship between force and velocity associated the user during a running activity;
assigning, based on the FVP, the user in a group of the plurality of groups;
generating a load profile of the user by changing load conditions of the user during the running activity and analyzing velocity changes for the load condition, the load profile indicative of a decrease in velocity of the user for different load conditions;
determining, based on the grouping and the load profile of the user, a training program to improve performance of the running activity of the user, the training program comprising one or more physical activities to be performed for a specified number of times using specified weights; and
instructing the user to perform a physical activity of the one or more physical activities from the training program.

8. The method of claim 7 wherein the force ratio is a ratio of a horizontal force and a vertical force exerted by the user during the running activity.

9. The method of claim 7, wherein the plurality of groups comprises:
a first group having the force ratio value of less or equal to 45%, the first group indicative of a substantially below average acceleration at the start of the running activity;
a second group having the force ratio value between 45% to 50%, the second group indicative of a below average acceleration at the start of the running activity;
a third group having the force ratio value of more than 50% and approximately 50% of an acceleration-velocity ratio indicating a low force, the third group indicative of a force-based power generated at the start of the running activity; and
a fourth group having the force ratio value of more than 50% and approximately 50% of an acceleration-velocity ratio indicating a low velocity, the fourth group indicative of a velocity-based power generated at the start of the running activity.

10. The method of claim 7, wherein determining the load profile comprises:
instructing the user to slide a first load, a second load, a third load, or a fourth load, during the running activity, each load being characterized by a percentage of the body weight of the user.

11. The method of claim 10, wherein determining the load profile comprises:
instructing the user to slide a load of approximately 10% of the body weight during the running activity;
instructing the user to slide a load in a range between 10% to 25% of the body weight during the running activity;
instructing the user to slide a load in a range between 25% to 50% of the body weight during the running activity; and
instructing the user to slide a load in a range between 75% to 85% of the body weight during the running activity.

12. A non-transitory computer-readable medium comprising instructions that, when executed by one or more processors, cause operations comprising:
receiving the FVP of the user, and a plurality of groups characterized by a force ratio of forces exerted during the running activity;
assigning, based on the FVP, the user in a group of the plurality of groups;
generating a load profile of the user by changing load conditions of the user during the running activity and analyzing velocity changes for the load condition, the load profile indicative of a decrease in velocity of the user for different load conditions;
determining, based on the grouping and the load profile of the user, a training program to improve performance of the running activity of the user, the training program comprising one or more physical activities to be performed for a specified number of times using specified weights; and
instructing the user to perform a physical activity of the one or more physical activities from the training program.

13. The medium of claim 1, wherein the force ratio is a ratio of a horizontal force and a vertical force exerted by the user during the running activity.

14. The medium of claim 1, wherein the plurality of groups comprises:
a first group having the force ratio value of less or equal to 45%, the first group indicative of a substantially below average acceleration at the start of the running activity;
a second group having the force ratio value between 45% to 50%, the second group indicative of a below average acceleration at the start of the running activity;
a third group having the force ratio value of more than 50% and approximately 50% of an acceleration-velocity ratio indicating a low force, the third group indicative of a force-based power generated at the start of the running activity; and
a fourth group having the force ratio value of more than 50% and approximately 50% of an acceleration-velocity ratio indicating a low velocity, the fourth group indicative of a velocity-based power generated at the start of the running activity.

15. The medium of claim 1, wherein determining the load profile comprises:
instructing the user to slide a first load, a second load, a third load, or a fourth load, during the running activity, each load being characterized by a percentage of the body weight of the user.

16. The medium of claim 15, wherein determining the load profile comprises:
instructing the user to slide a load of approximately 10% of the body weight during the running activity;
instructing the user to slide a load in a range between 10% to 25% of the body weight during the running activity;
instructing the user to slide a load in a range between 25% to 50% of the body weight during the running activity; and
instructing the user to slide a load in a range between 75% to 85% of the body weight during the running activity.
